(19) United States
(12) Patent Application Publication
(10) Pub. No.: US 2018/0015420 A1
(43) Pub. Date: Jan. 18, 2018

(54) Method for the Pervaporation and Vapor-Permeation Separation of Gas-Liquid Mixtures and Liquid Mixtures by SAPO-34 Molecular Sieve Membrane

(71) Applicants: Shanghai Advanced Research Institute, Chinese Academy of Sciences, Shanghai (CN); Shanxi Lu'An Environmental Protection & Energy Development Co., Ltd., Changzhi (CN); Total Raffinage Chimie, Courbevoie (FR)

(72) Inventors: Daniel Curulla-Ferre, Uccle (BE); Yuhan Sun, Shanghai (CN); Zhiqiang Sun, Changzhi (CN); Yanfeng Zhang, Shanghai (CN)

(21) Appl. No.: 15/547,867

(22) PCT Filed: Feb. 2, 2016
(86) PCT No.: PCT/EP2016/052209

§ 371 Date: Aug. 1, 2017

(30) Foreign Application Priority Data
Feb. 3, 2015 (CN) 201510054331.8

(51) Int. Cl. B01D 61/36; C07C 29/76; C01B 39/54; B01D 67/00; B01D 19/00; B01D 71/02; B01D 69/10; C07C 68/08; C01B 37/08

(52) U.S. Cl. CPC B01D 61/362; C07C 68/08; C07C 29/76; C01B 39/54; C01B 37/08; B01D 19/0031; B01D 71/028; B01D 69/105; B01D 67/0051; B01D 2323/46

(57) ABSTRACT

The present invention discloses a method for the pervaporation and vapor-permeation separation of a gas-liquid mixture or a liquid mixture by a SAPO-34 molecular sieve membrane, which comprises: 1) mixing an Al source, tetraethyl ammonium hydroxide, water, a Si source and a P source, and subjecting the resultant to hydrothermal crystallization, then centrifuging, washing and drying to get SAPO-34 molecular sieve seeds; 2) coating the SAPO-34 molecular sieve seeds onto the inner surface of a porous support tube; 3) synthesis of a SAPO-34 molecular sieve membrane tube; 4) calcining the obtained SAPO-34 molecular sieve membrane tube to obtain a SAPO-34 molecular sieve membrane; 5) using the SAPO-34 molecular sieve membrane obtained from step 4) to perform separation of a gas-liquid mixture or a liquid mixture via a process of pervaporation separation or vapor-permeation separation. The invention has the advantages of very high methanol selectivity and permeation flux, and provides an efficient and energy-saving separation way via pervaporation or vapor-permeation separation.

### FIELD OF THE INVENTION

The invention relates to a method for the separation of a mixture by using a SAPO-34 molecular sieve membrane, especially to a method for the separation of a gas-liquid mixture or a liquid mixture through pervaporation (pervaporative separation) or vapor-permeation by a SAPO-34 molecular sieve membrane.

### BACKGROUND OF THE INVENTION

Dimethyl carbonate (DMC), which has a molecular formula of CO(OCH₃)₂, is a good solvent, has low volatility and similar toxicity values to anhydrous ethanol, and is completely biodegradable. It is an environmental-friendly chemical and finds extensive applications in the fields of pharmaceutical, chemical engineering and energy etc. DMC molecules have an oxygen content of 53%, which is three times higher than that of methyl tert-butyl ether (MTBE). It can be used as an additive in gasoline to enhance octane number and to suppress emission of carbon monoxide and hydrocarbons. It is very active in terms of chemistry, and it is an important intermediate and starting material for organic synthesis and, thus, it is known as a new foundation of organic synthesis.

The industrial methods for producing DMC mainly include methods of oxidative carbonylation, transesterification, or phosgenation of methanol [Appl. Catal. A Gen., 221(2001) 241-251]. No matter which method is used, a mixture of methanol (MeOH) and DMC was always obtained from the reactions. At normal pressure, MeOH and DMC would form a binary azeotrope (70 wt % MeOH and 30 wt % DMC), whose azeotropic temperature is 64° C. Therefore, it is necessary to separate and recover DMC from the azeotrope. Currently, methods for separation of the MeOH/DMC azeotrope mainly include low temperature crystallization, adsorption, extractive distillation, azeotropic distillation and pressure distillation. All of these methods possess the disadvantages and shortcomings that energy consumption is high, it is difficult to select the appropriate solvent, it is difficult to operate and the safety has deficiencies. In contrast, a membrane separation method possesses advantages of low energy consumption, high efficiency and flexible operation conditions.

Membrane separation technology uses the differential chemical potential of a component on both sides of the membrane as a driving force. The membrane can be used to achieve selective separation of different components in feed liquids according to different affinity and mass transfer resistance of the components. Membrane materials can be classified as polymeric membrane, inorganic membrane and composite membrane. In recent years, some progress has been made in studies on separation of a MeOH/DMC mixture using membrane technologies, which mainly focus on polymeric membranes. It was found that materials such as polyvinyl alcohol (PVA), polyacrylic acid (PAA), chitosan or the like can be prepared into pervaporation membranes which preferentially remove methanol and have good separation performance.

Wooyoung et al. used a cross-linked chitosan membrane for pervaporation separation of MeOH/DMC and investigated the influences of operation temperature and feed composition on the separation factor and flux and received a good result [Separation and Purification Technology 31 (2003) 129-140]. Wang et al. prepared a PAA/PVA mixed membrane, wherein a mixed membrane containing 70 wt % PPA has a separation factor of 13 and a permeation flux of 577 g/(m² h) [Journal of Membrane Science 305 (2007) 238-246]. Pasternak et al. tested the performance of a PVA membrane for the separation of MeOH/DMC. The MeOH concentration on the permeate side is concentrated from 70 wt % on the feed side to 93-97 wt % and the flux was 110-1130 g/(m² h) [U.S. Pat. No. 4,798,674 (1989)]. Chen et al. prepared a hybrid membrane of chitosan and silica through cross-linking chitosan with aminopropyl triethoxy silane. Separation factor of 30 and permeation flux of 1265 g/(m² h) were achieved at 50° C. for a 70/30 MeOH/DMC mixture.

However, the polymer membrane faced so many problems that affected its separation performance and application range. For instance, during separation, a swelling phenomenon would occur, the chemical stability degrades, especially mechanical strength and thermal stability degrades, which limit its application under severe conditions such as high pressure and high temperature. On the other hand, the inorganic membranes, typically of a molecular sieve type, can well solve these issues because the inorganic membranes have uniform pore size for separation and good thermal, mechanical and chemical stability. Therefore, the inorganic membranes can be used for separation in an environment under harsh conditions such as high temperature and high pressure. Thus, it becomes possible to carry out the vapor phase separation of a liquid mixture under conditions of relative high temperature and pressure by using a molecular sieve membrane. However, currently the main application of molecular sieve membranes is dehydration of organics. Applications of a molecular sieve membrane in the separation, especially vapor phase separation at high temperature, of a MeOH/DMC mixture were rarely reported. Pina et al. synthesized a NaA molecular sieve membrane on Al₂O₃ support and used the NaA molecular sieve membrane to separate a water/ethanol mixture by pervaporation, in which the separation factor can reach 3600 and the permeation flux of water reaches 3800 g/(m² h)[Journal of Membrane Science 244 (2004) 141-150]. Hidetoshi et al. studied pervaporation separation performance of NaX and NaY molecular sieve membranes. It was found that the membranes have very high selectivity to alcohols and benzene. They also studied the selectivity of these membranes for MeOH/DMC separation, and as a result, separation factor of 480 and permeation flux of 1530 g/(m² h) were achieved while the feed composition was 50/50 [Separation and Purification Technology 25 (2001) 261-268].

### SUMMARY OF THE INVENTION

The technical problem to be solved by the present invention is to provide a method for the separation of a gas-liquid mixture or a liquid mixture by pervaporation and vapor-permeation through a SAPO-34 molecular sieve membrane. The present invention mainly provides a method for synthesizing a SAPO-34 molecular sieve membrane and separating a gas-liquid mixture or a liquid mixture by pervaporation and vapor-permeation through the resultant SAPO-34 molecular sieve membrane. The prepared high-performance SAPO-34 molecular sieve membrane can be used in pervaporation or vapor-permeation separation of a mixture (e.g. MeOH/DMC). Moreover, the inventive method achieves a very high methanol (MeOH) selectivity and permeation flux. It also has the advantages of high efficiency and saving energy.

To resolve the issues mentioned above, the invention provides a method for pervaporation or vapor-permeation separation of a gas-liquid mixture or a liquid mixture (e.g. separation of a methanol-containing mixture) by a SAPO-34 molecular sieve membrane, which includes the following steps:

1) mixing and dissolving an Al source, tetraethylammonium hydroxide (TEAOH), water, a Si source and a P source to make a reaction liquor for seeds (crystal seeds), which is then subjected to crystallization for 4-7 h by heating at 170-210° C. (i.e., hydrothermal crystallization), then centrifuging, washing and drying to get SAPO-34 molecular sieve seeds;

wherein the molar ratio of the Al source, P source, Si source, tetraethylammonium hydroxide and all water in the reaction liquor for seeds is:1 Al₂O₃: 1-2 P₂O₅: 0.3-0.6 SiO₂:1-3 TEAOH: 55-150 H₂O;

2) coating the SAPO-34 molecular sieve seeds onto the inner surface of a porous support tube to get a porous support tube coated with SAPO-34 molecular sieve seeds;

3) the synthesis of SAPO-34 molecular sieve membrane tube

A. uniformly mixing an Al source, a P source, a Si source, tetraethylammonium hydroxide (TEAOH), di-n-propyl amine (DPA), water and a fluoride to form a mother liquor for molecular sieve membrane synthesis;

wherein the molar ratio of the Al source, P source, Si source, tetraethylammonium hydroxide, di-n-propyl amine and all water in the mother liquor for molecular sieve membrane synthesis is 1 Al₂O₃:0.5-3.5 P₂O₅:0.05-0.6 SiO₂:0.5-8 TEAOH: 0.1-4.0 DPA: 0.01-1F⁻: 50-300 H₂O;

B. placing the porous support tube coated with SAPO-34 molecular sieve seeds obtained from step 2) in the mother liquor for molecular sieve membrane synthesis and after aging for 2-8 h at room temperature −80° C., crystallizing for 3-24 h at 150-240° C. to synthesize the SAPO-34 molecular sieve membrane tube;

4) calcination to remove the template agent calcining the SAPO-34 molecular sieve membrane tube obtained in step 3) at 370-700° C. for 2-8 h, to get a SAPO-34 molecular sieve membrane tube having the template agent (tetraethyl ammonium hydroxide) removed;

5) using the SAPO-34 molecular sieve membrane obtained in step 4) to perform separation of a gas-liquid mixture or a liquid mixture by a process of pervaporation separation or vapor-permeation separation. The gas in the gas-liquid mixture includes common gases, for example includes inert gas, hydrogen gas, oxygen gas, CO₂ or gaseous hydrocarbon, and the liquid in the gas-liquid mixture includes common solvents such as water, alcohol, ketone or aromatics;

Wherein in the step 5), the inert gas contains N₂;

the gaseous hydrocarbon contains methane;

the alcohol contains methanol, ethanol, or propanol;

the ketone contains acetone or butanone;

the aromatics contain benzene.

In addition, in the step 5), in the separation of the liquid mixture by the SAPO-34 molecular sieve membrane, said liquid mixture is a mixture of methanol and a liquid other than methanol, said liquid other than methanol includes one of dimethyl carbonate, ethanol, methyl tert-butyl ether. In the step 1, the detailed preparation method for the reaction liquor for seeds can be operated as follows: adding the Al source to the tetraethylammonium hydroxide TEAOH solution, and after hydrolysis, adding the Si source and then the P source, stirring, to get the reaction liquor for seeds.

More specifically, the operation can be as follows: mixing the tetraethylammonium hydroxide solution with DI water, then adding the Al source to the resultant solution, stirring for 2-3 h at room temperature; then adding the Si source dropwise, stirring for 0.5-2 h; then slowly adding the P source dropwise, stirring for 12-24 h, thereby to get the reaction liquor for seeds. In the steps 1) and 3), the Al source includes one or more of aluminum isopropoxide, Al(OH)₃, elemental aluminum, an Al salt; wherein said Al salt includes one or more of aluminum nitrate, aluminum chloride, aluminum sulfate , and aluminum phosphate.

In the steps 1) and 3), the P source includes phosphoric acid.

In the steps 1) and 3), the Si source includes one or more of tetraethyl orthosilicate (TEOS), tetramethyl orthosilicate (TMOS), silica sol, silica, sodium silicate, and water glass.

In the step 1), the heating is preferably microwave heating. In step 1), the size of the SAPO-34 molecular sieve seeds is 50-1000 nm.

In the step 2), the porous support tube includes a porous ceramic tube; wherein the pore size of the porous ceramic tube is 5-2000 nm, and the material of the tube is selected from Al₂O₃, TiO₂, ZrO₂, SiC or silicon nitride.

In the step 2), the detailed procedure for the coating of the seeds is: sealing the two ends of the porous ceramic tube with glaze, washing and drying, sealing the outer surface, and then coating the SAPO-34 molecular sieve seeds onto the inner surface of the porous support tube. The coating method includes brush coating or dip coating.

In the step 3), the fluoride includes one or a mixture of: HF, and a fluoride salt; wherein the fluoride salt includes ammonium fluoride, a fluoride salt of a main-group metal or a fluoride salt of a transition metal. Preferably, said fluoride salt includes one or more of sodium fluoride, potassium fluoride and ammonium fluoride.

In the step 3), the operation procedures of forming the mother liquor for the molecular sieve membrane synthesis are as follows: mixing the Al source, P source and water, stirring for 1-5 h; then adding the Si source, stirring for 0.5-2 h; then adding tetraethyl ammonium hydroxide, stirring for 0.5-2 h; then adding di-n-propylamine, stirring for 0.5-2 h; then adding the fluoride, stirring for 12˜96 h at room temperature—60° C., thereby to get a homogeneous mother liquor for molecular sieve membrane synthesis.

In the step 4), the atmosphere for calcination is selected from: inert gas, vacuum, air, oxygen gas, or diluted oxygen gas in any ratio. In the calcination, the temperature increasing rate and the temperature decreasing rate were not higher than 2K/min.

In the step 5), the conditions for the process of pervaporation separation or vapor-permeation separation are: methanol concentration in the feed: 1-99 wt % (mass percentage), permeation operation temperature: room temperature −150° C., feed pressure: atmospheric pressure ˜20 atms, pressure on the permeate side: 0.06-2000 Pa, feed flow rate: 1-500 mL/min.

This invention provides a process of pervaporation separation or vapor-permeation separation, wherein a SAPO-34 molecular sieve membrane is used for separation of a gas-liquid mixture or a liquid mixture, e.g. MeOH/DMC (mixture. When the operation temperature and pressure were 120° C. and 0.3 MPa, respectively, the separation factor for separating a MeOH/DMC (70:30) azeotrope by the SAPO-34 molecular sieve membrane was above 1000, and the resultant methanol concentration was above 99.99 wt %. Thus, this invention provides a high efficiency, energy saving method for separation of a methanol/dimethyl carbonate (DMC) mixture. Therefore, the membrane separation method of MeOH/DMC has advantages like low energy consumption, being not limited by azeotropic mixture, high methanol flux and high separation factors, and thus has great economic value.

Besides the separation of MeOH/DMC mixture, the SAPO-34 molecular sieve membrane of the present invention could also be used for pervaporation or vapor-permeation separation of a mixture of methanol with other liquid, such as methanol-ethanol, methanol-methyl tert-butyl ether (MTBE).

In addition, the SAPO-34 molecular sieve membrane of the present invention can also be used for pervaporation or vapor-permeation separation of a gas-liquid mixture.

### BRIEF DESCRIPTION OF THE DRAWINGS

The invention will be explained in further detail by taking the appended figures and the examples.

FIG. 1. is a SEM (Scanning Electron Microscopy) image of SAPO-34 seeds of Example 1.

FIG. 2. is an XRD (X-ray diffraction) pattern of SAPO-34 seeds of Example 1.

FIG. 3. is a surface SEM image of SAPO-34 molecular sieve membrane of Example 1 (prepared by adding 0.1 mol HF).

FIG. 4. is a cross sectional SEM image of SAPO-34 molecular sieve membrane of Example 1 (prepared by adding 0.1 mol HF).

FIG. 5. is a schematic diagram of a pervaporation separation process, wherein 1 denotes feed liquid, 2 denotes peristaltic pump, 3 denotes molecular sieve membrane assembly and heat source, 4 denotes stop valve, 5 denotes cold trap, 6 denotes vacuum gauge, 7 denotes vacuum pump.

FIG. 6. is a surface SEM image of SAPO-34 molecular sieve membrane of Example 4 (prepared by adding 0.1 mol NH₄F).

FIG. 7. is a cross sectional SEM image of SAPO-34 molecular sieve membrane of Example 4 (prepared by adding 0.1 mol NH₄F).

### EXAMPLES

### Example 1

### Separation of Methanol/Dimethyl Carbonate at Different Feed Composition by SAPO-34 Molecular Sieve Membrane

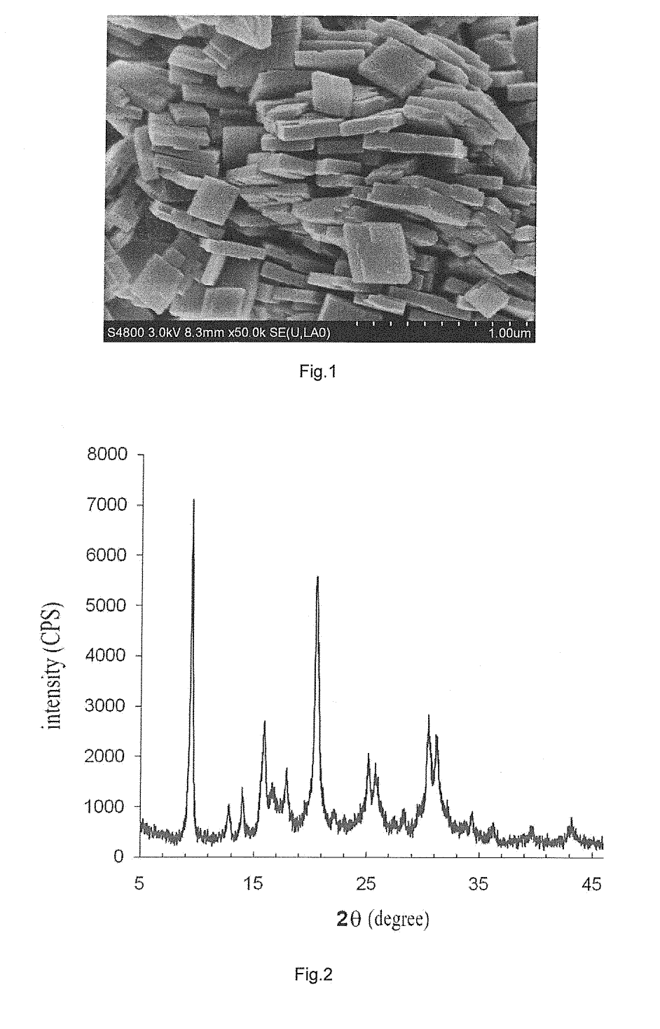

Step1: 2.46 g of DI water were added to 31.13 g of tetraethyl ammonium hydroxide solution (TEAOH, 35 wt %) . Then 7.56 g of aluminum isopropoxide were added thereto, and the resultant was stirred for 2-3 h at room temperature. Then 1.665 g of silica sol (40 wt %) were added dropwise and the resultant was stirred for 1 h. Finally, 8.53 g of phosphoric acid solution (H₃PO₄, 85 wt %) were added slowly dropwise and the resultant was stirred overnight (e.g., stirred for 12 hours). Then crystallization was performed at 180° C. for 7 h by using microwave heating. The obtained product was taken out from the reactor, centrifuged, washed, dried, to obtain SAPO-34 molecular sieve seeds. The SEM image of the seeds is shown in FIG. 1 and the XRD pattern of the seeds is shown in FIG. 2. From the SEM image, it can be seen that the size of the seeds is around 300 nm*300 nm*100 nm. Moreover, the XRD pattern indicates that the seeds are pure SAPO-34 phase, and are well crystallized with no impure phase.

Step 2: A porous ceramic tube (material: alumina) with 5 nm pore size was used as a support. The two ends of the support were sealed with glaze. After washing and drying, the out surface of the support was sealed (covered) by PTFE tape. Then the SAPO-34 molecular sieve seeds were coated onto the inner surface of the ceramic tube by brush coating method. Thus, a porous ceramic tube coated with SAPO-34 molecular sieve seeds was obtained.

Step 3: 4.27 g of phosphoric acid solution (H₃PO₄, 85 wt %) were mixed with 43.8 g of DI water, and the resultant was stirred for 5 min. Then 7.56 g of aluminum isopropoxide were added, and the resultant was stirred for 3 h at room temperature. 0.83 g of silica sol (40 wt %) were added, and the resultant was stirred for 30 min at room temperature. Then, 7.78 g of tetraethyl ammonium hydroxide solution (TEAOH, 35 wt %) were added dropwise, and the resultant was stirred for 1 h at room temperature. Finally, 3.0 g of di-n-propylamine were added thereto, and after the resultant was stirred for 30 min at room temperature. 0.045 g of hydrofluoric acid (HF, 40 wt %) were added, and the resultant was stirred overnight (e.g., stirred for 12 hours) at 5° C., getting a uniform mother liquor for synthesis of SAPO-34 molecular sieve membrane. The porous ceramic tube coated with SAPO-34 molecular sieve seeds, which was prepared in the above step 2, was placed in a reaction vessel, and the mother liquor for synthesis of SAPO-34 molecular sieve membrane was added. The reaction vessel was closed and aging was performed for 3 h at room temperature. Then hydrothermalsynthesis was performed at 22° C. for 5 h. After taken out from the reaction vessel, the product was thoroughly rinsed and dried in an oven. Thus, a SAPO-34 molecular sieve membrane tube was obtained.

Step 4: The SAPO-34 molecular sieve membrane tube obtained in step 3 was calcined in vacuum for 4 h to remove the template agent (the temperature increasing rate and temperature decreasing rates were 1° C./min, respectively), thereby to obtain an activated SAPO-34 molecular sieve membrane.

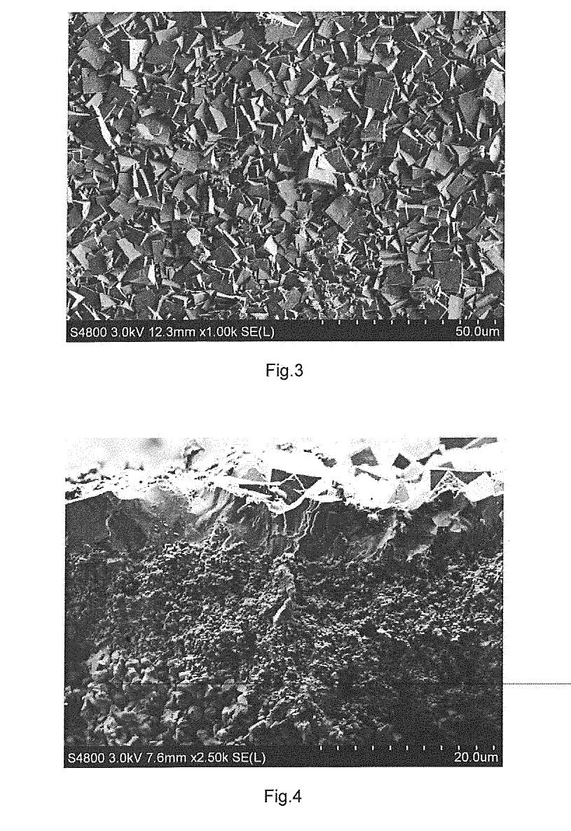

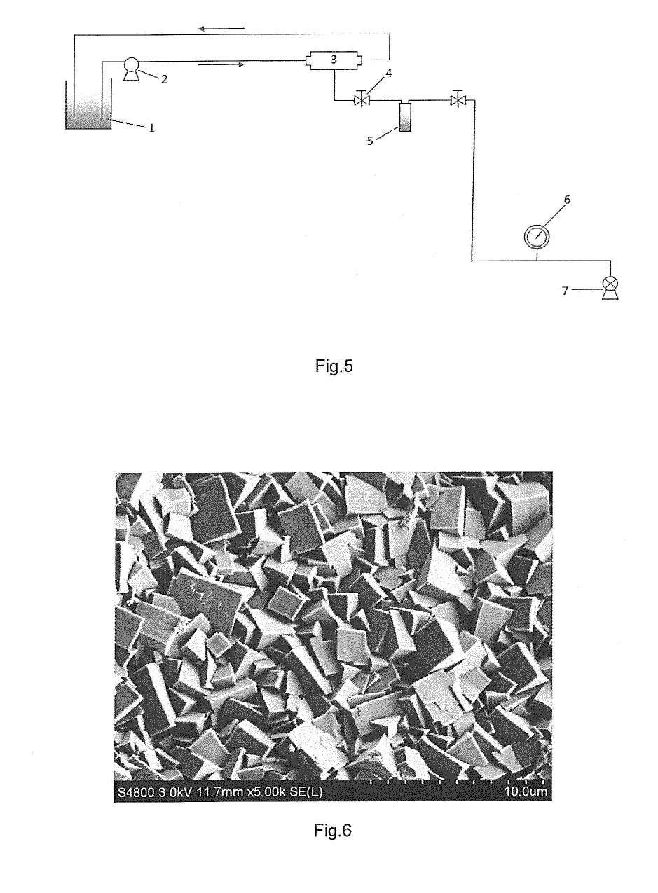

The surface and cross sectional SEM images of the SAPO-34 molecular sieve membrane (prepared by addition of 0.1 mol HF) are respectively shown in FIGS. 3 and 4. It can be seen that the support surface is completely covered by square lamellar SAPO-34 crystals which are perfectly cross-linked therebetween. The crystal size is 4-7 microns, and the molecular sieve membrane surface is flat. The cross sectional image shows that the thickness of the membrane is about 5-6 microns.

Step 5. A methanol/dimethyl carbonate (i.e., DMC/MeOH) azeotrope was separated by pervaporation separation process at a permeation operation temperature of 120° C., a feed pressure of 0.3 MPa, a feed flow rate of 1 mL/min, a pressure on the permeate side of 100 Pa, with a composition (in mass ratio) of the MeOH/DMC feed being 90/10, 70/30, 50/50, 30/70 and 10/90, respectively. The schematic diagram of the pervaporation process is shown in FIG. 5.

The separation factor is calculated from: α=(w₂ₘ/w_{2d})/(w₁ₘ/w_{1d}), where w₂ₘ is the mass concentration of methanol on the permeate side, w_{2d} is the mass concentration of dimethyl carbonate on the permeate side, w₁ₘ is the mass concentration of methanol in the feed and w_{1d} is the mass concentration of dimethyl carbonate (DMC) in the feed.

The permeation flux equation is J=Δm/(s×t), wherein Δm is the mass (g) of a product collected on the permeate side, s is the molecular sieve membrane area (m²) and t is the collecting time (h).

TABLE 1
The pervaporation separation test results of MeOH/DMC in Example 1.

| Feed | | | Methanol concentration in |
|---|---|---|---|
| composition | Permeation flux J | Separation | the permeated product |
| MeOH/DMC | [g/(m² · h)] | factor α | (wt %) |
| 10/90 | 94 | 5600 | 99.840 |
| 30/70 | 168 | 2620 | 99.911 |
| 50/50 | 384 | 5000 | 99.980 |
| 70/30 | 806 | 8600 | 99.995 |
| 90/10 | 1498 | 5300 | 99.998 |

It can be seen from Table 1 that at different feed compositions, the SAPO-34 molecular sieve membranes synthesized from the fluoride-containing system have very high methanol selectivity.

The separation factor reaches a minimum of 2620 when the feed has a composition of 30-70 wt %, and reaches a maximum of about 8600 when the feed has a composition of 70-30 wt %. The methanol concentration in the permeate is at least 99.84 wt %. With the increase of methanol concentration in the feed, the permeation flux gradually increases, which is caused by the increasing of methanol partial pressure.

### Example 2

### Separation of Methanol/Dimethyl Carbonate by SAPO-34 Molecular Sieve Membrane at Different Operation Temperatures

All steps in this Example are the same as in Example 1 except that in step 5, the feed composition of MeOH/DMC is 90/10, and the operation temperature is 100° C., 110° C., 120° C., 130° C., 140° C., respectively.

TABLE 2
The vapor-permeation separation test
results of MeOH/DMC in Example 2.

| Operation | | |
|---|---|---|
| temperature | Methanol permeation flux J | Separation |
| ° C. | [kg/(m² · h)] | factor α |
| 100 | 0.71 | 1300 |
| 110 | 0.92 | 1330 |
| 120 | 1.10 | 5300 |
| 130 | 1.80 | 3800 |
| 140 | 2.00 | 3450 |

It can be seen from Table 2 that at different operation temperatures (100-140° C.), the SAPO-34 molecular sieve membranes synthesized from the fluoride-containing system have very high methanol selectivity. With the increase of operation temperature, the permeation flux of methanol gradually increases, which is due to the increase of methanol partial pressure.

### Example 3

### Separation of Methanol/Dimethyl Carbonate by SAPO-34 Molecular Sieve Membrane at Different Feed Pressures

All steps in this Example are the same as in Example 1 except that in step 5, the feed composition of MeOH/DMC is 90/10, and the feed pressures are 0.3 MPa, 0.4 MPa, 0.5 MPa, 0.6 MPa, respectively.

TABLE 3
The pervaporation separation test results of MeOH/DMC in Example 3.

| Feed pressure | Methanol permeation flux J | Separation |
|---|---|---|
| MPa | [kg/(m² · h)] | factor α |
| 0.6 | 1.65 | 3050 |
| 0.5 | 1.68 | 2720 |
| 0.4 | 1.30 | 3100 |
| 0.3 | 1.10 | 5300 |

It can be seen from Table 3 that at different feed pressures, the SAPO-34 molecular sieve membrane synthesized from the fluoride-containing system have very high methanol selectivity. With the increase of system pressure, the permeation flux increases gradually. When the pressure reaches 0.5 MPa, the methanol permeation flux becomes constant.

### Example 4

### Separation of Methanol/Dimethyl Carbonate by SAPO-34 Molecular Sieve Membrane Synthesized by Addition of Different Fluoride

All steps in this Example are the same as in Example 1 except that in step 3, 0.037 g of sodium fluoride, 0.033 g of ammonium fluoride are added respectively, and in step 5, the feed composition of MeOH/DMC is 90/10, and the feed pressure is 0.3 MPa.

TABLE 4
The pervaporation separation test results of MeOH/DMC in Example 4.

| | Methanol permeation flux J | Separation |
|---|---|---|
| Fluoride | [kg/(m² · h)] | factor α |
| NaF | 1.03 | 4200 |
| NH₄F | 1.14 | 3900 |

It can be seen from Table 4 that the SAPO-34 molecular sieve membranes synthesized from the system containing a different fluoride have very high methanol selectivity and high permeation flux. Thus, in case of addition of ammonium fluoride and sodium fluoride, a high-performance SAPO-34 molecular sieve membranes can also be prepared.

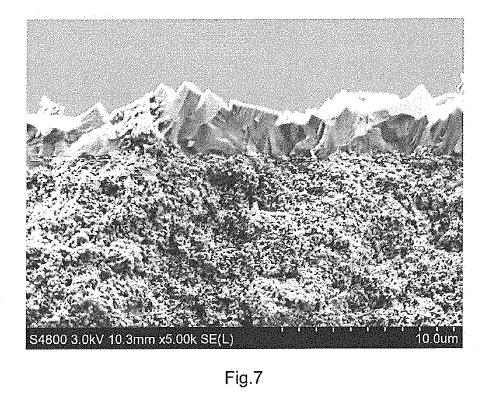

The surface and sectional SEM images of the SAPO-34 molecular sieve membrane (prepared by adding 0.1 mol NH4F) are respectively shown in FIGS. 6 and 7. It can be seen that the support surface was completely covered by square lamellar SAPO-34 crystals which are perfectly cross-linked therebetween. The crystal size is 4-7 microns, and the molecular sieve membrane surface is flat. The images of the cross section show that the thickness of the membrane is about 5-6 microns.

In addition, the SAPO-34 molecular sieve membranes prepared as above can also be used for the pervaporation or vapor-permeation separation of a gas-liquid mixture, wherein the gas of the gas-liquid mixture may be one of nitrogen gas, hydrogen gas, oxygen gas, carbon dioxide or methane or the like. The liquid of the gas-liquid mixture may be one of water, methanol, acetone or benzene or the like.

1. A method for pervaporation separation of a gas-liquid mixture or a liquid mixture by preparing and using a SAPO-34 molecular sieve membrane, characterized in that the method comprises:
1) mixing and dissolving an Al source, tetraethyl ammonium hydroxide TEAOH, water, a Si source and a P source to make a reaction liquor for seeds, which is then subjected to crystallization for 4-7 h by heating at 170-210° C., then centrifuging, washing and drying to get SAPO-34 molecular sieve seeds;
wherein the molar ratio of the Al source, P source, Si source, tetraethylammonium hydroxide and all water in the reaction liquor for seeds is 1 Al₂O₃: 1-2 P₂O₅: 0.3-0.6 SiO₂: 1-3 TEAOH: 55-150 H₂O.
2) coating the SAPO-34 molecular sieve seeds onto the internal surface of a porous support tube to get a porous support tube coated with SAPO-34 molecular sieve seeds;
3) synthesizing a SAPO-34 molecular sieve membrane tube by:
A. uniformly mixing an Al source, a P source, a Si source, tetraethylammonium hydroxide TEAOH, di-n-propyl amine DPA, water and a fluoride to form a mother liquor for molecular sieve membrane synthesis;
wherein the molar ratio of the Al source, P source, Si source, tetraethylammonium hydroxide, di-n-propyl amine and all water in the mother liquor for molecular sieve membrane synthesis is 1 Al₂O₃: 0.5-3.5 P₂O₅: 0.05-0.6 SiO₂: 0.5-8 TEAOH: 0.1-4.0 DPA: 0.01-1F⁻: 50-300 H₂O;
B. placing the porous support tube coated with SAPO-34 molecular sieve seeds obtained from step 2) in the mother liquor for molecular sieve membrane synthesis and after aging for 2-8 h at room temperature −80° C., crystallizing for 3-24 h at 150-240° C. to synthesize the SAPO-34 molecular sieve membrane tube;
4) calcining the SAPO-34 molecular sieve membrane tube obtained in step 3) at 370-700° C. for 2-8 h, to get a SAPO-34 molecular sieve membrane;
5) using the SAPO-34 molecular sieve membrane obtained in step 4) to perform the separation of a liquid mixture via a process of pervaporation separation;
wherein the liquid mixture is a mixture of methanol and a liquid other than methanol, wherein said liquid other than methanol includes one of dimethyl carbonate, ethanol, methyl t-butyl ether.

2. The method according to claim 1 characterized in that in steps 1) and 3), the Al source includes one or more of aluminum isopropoxide, Al(OH)₃, elemental aluminum, an Al salt; wherein said Al salt includes one or more of aluminum nitrate, aluminum chloride, aluminum sulfate, and aluminum phosphate;
in steps 1) and 3), the P source includes phosphoric acid; and
in steps 1) and 3), the Si source includes one or more of tetraethyl orthosilicate, tetramethyl orthosilicate, silica sol, silica, sodium silicate, and water glass.

3. The method according to claim 1, characterized in that in step 1), the heating comprises microwave heating; and the size of the SAPO-34 molecular sieve seeds is 50-1000 nm.

4. The method according to claim 1 characterized in that in step 2), the porous support tube includes a porous ceramic tube; wherein the pore size of the porous ceramic tube is 5-2000 nm; and the material of the porous ceramic tubes is selected from Al₂O₃, TiO₂, ZrO₂, SiC or silicon nitride.

5. The method according to claim 1 characterized in that the coating of the seeds in step 2), is performed according to the following procedure,
sealing the two ends of the porous support tube with glaze, washing and drying, sealing the outer surface, and then coating the SAPO-34 molecular sieve seeds onto the inner surface of the porous support tube; wherein the coating method includes brush coating or dip coating.

6. The method according to claim 1 characterized in that in step 3), the fluoride includes one or a mixture of HF, and a fluoride salt; wherein the fluoride salt includes ammonium fluoride, a fluoride salt of a main-group metal or a fluoride salt of a transition metal.

7. The method according to claim 6, characterized in that the fluoride salt includes one or more of potassium fluoride, sodium fluoride, and ammonium fluoride.

8. The method according to claim 1, characterized in that in step 3), the operation procedures of forming the mother liquor for molecular sieve membrane synthesis are as follows,
mixing the Al source, P source and water, stirring for 1-5 h; then adding the Si source, stirring for 0.5-2 h; then adding tetraethyl ammonium hydroxide, stirring for 0.5-2 h; then adding di-n-propyl amine, stirring for 0.5 h; then adding the fluoride, stirring for 12-96 h at room temperature—60° C., thereby to get a homogeneous mother liquor for molecular sieve membrane synthesis.

9. The method according to claim 1 characterized in that in step 4) the atmosphere for calcination is selected from inert gas, vacuum, air, oxygen gas, or diluted oxygen gas in any ratio; and in the calcination, the temperature increasing rate and the temperature decreasing rate are not higher than 2K/min.

10. The method according to claim 1 characterized in that in step 5), the conditions for the process of pervaporation separation are: a methanol concentration in the feed of 1-99 wt %, a permeation operation temperature ranging from 20°C. to 150° C., a feed pressure ranging from atmospheric pressure to 20 atms, a pressure on the permeate side ranging from 0.06 Pa to 2000 Pa, and a feed flow rate ranging from 1-500 mL/min;
